(19) United States
(12) Patent Application Publication
(10) Pub. No.: US 2023/0330026 A1
(43) Pub. Date: Oct. 19, 2023

(54) Large Fast Dispersing Tablet Prepared By Lyophilization

(71) Applicant: Navinta III Inc, Boca Raton, FL (US)

(72) Inventors: Mahendra R. Patel, Delray Beach, FL (US); Sandipkumar Arvindbhai Patel, Robbinsville, NJ (US)

(21) Appl. No.: 18/015,482

(22) PCT Filed: Jul. 9, 2021
(86) PCT No.: PCT/US2021/041061

§ 371 Date: Jan. 10, 2023

Related U.S. Application Data
(60) Provisional application No. 63/050,439, filed on Jul. 10, 2020.

(51) Int. Cl. A61K 9/20; A61K 9/19; A61J 1/03

(52) U.S. Cl. CPC A61K 9/2095; A61K 9/19; A61J 1/035

(57) ABSTRACT

A tablet formed from a pre-lyophilization solution has a void in its bottom surface, with a void height that is about 60% to 100% of a depth of the tablet. A pharmaceutical dosage form includes a blister sheet having one or more cavities, each having a protrusion extending upwardly from the cavity floor, with a tablet formed in the cavity by lyophilization of a pre-lyophilization solution in each such cavity. The protrusion is a cylinder or tapered cylinder and the void has a corresponding shape. A film overlays the blister sheet to seal the tablet in the blister sheet cavities.

### FIELD OF THE INVENTION

The present invention relates to apparatus and methods for preparing oral and vaginal pharmaceutical dosage forms by a lyophilization process.

### BACKGROUND OF THE INVENTION

Lyophilization is used in preparing both sterile dosage forms and non-sterile pharmaceutical dosage forms. Lyophilization involves the removal of water or other solvents from a given product by a process called sublimation. This occurs when the ice of a frozen product converts directly to the gaseous state without passing through the liquid phase. It is the most commonly used process in the pharmaceutical industry where a product is heat sensitive to terminal sterilization and/or not stable in the solution state. In a typical lyophilization process, non-sterile solids are dissolved in solvent to form a solution. The solution is then aseptically filtered through a 0.2μ sterile grade filter. The filtered solution is then loaded into a suitable container which is processed in a lyophilizer chamber.

Lyophilization is typically performed in three consecutive steps by controlling the temperature of product/container containing product and the pressure of lyophilizer chamber. The typical steps are freezing, primary drying, and secondary drying. Lyophilized containers are then fully closed with a suitable closing element.

U.S. Pat. No. 6,413,549 discloses an oral solid, rapidly disintegrating, freeze-dried dosage form containing coarse particles of a pharmaceutically active material, which is uncoated or coated with a polymer or lipid material. The oral dosage form comprises coarse particles having a size in the range of 50 micron to 400 micron and disintegrates in the oral cavity in less than 10 seconds.

U.S. Pat. No. 9,192,580 discloses a pharmaceutical composition in an oral solid, molded fast-dispersing dosage form comprising an active ingredient and a non-hydrolyzed, non-gelling fish gelatin carrier which releases the active ingredient, wherein the composition disintegrates within 1 to 60 seconds of being placed in contact with fluid, and the dosage form comprises a network of the active ingredient and the non-hydrolyzed, non-gelling fish gelatin carrier. The dosage is prepared by subliming solvent from an admixture in the solid state in which the admixture comprises the active ingredient, the non-hydrolyzed, non-gelling fish gelatin carrier and a solvent. The patent also discloses a process for preparing fast-dispersing dosage forms by freeze-drying or lyophilizing a combination of the active ingredient and fish gelatin (e.g., non-gelling fish gelatin).

U.S. Pat. No. 5,595,761 to Allen Jr. et al. discloses a particulate support matrix for use in making a rapidly dissolving tablet, comprising a first polypeptide component having a net charge when in solution, e.g. non-hydrolyzed gelatin; a second polypeptide component having a net charge of the same sign as the net charge of the first polypeptide component when in solution e.g. hydrolyzed gelatin; and a bulking agent. The first polypeptide component and the second polypeptide component together comprise about 2% to 20% by weight of the particulate support matrix and the bulking agent comprises about 60% to 96% by weight of the particulate support matrix. The second polypeptide component has a solubility in aqueous solution greater than the first polypeptide component and the mass to mass ratio of the first polypeptide component to the second polypeptide component is from about 2:1 to about 1:14. The support matrix is said to disintegrate within less than about 20 seconds.

Published International Application No. WO 93/13758 (PCT/US92/07497) describes tablets of increased physical strength which are prepared by combining and compressing a meltable binder, excipients and a pharmaceutically active agent into a tablet, melting the binder into the tablet and then solidifying the binder. In one embodiment, a disintegrating agent is utilized to increase the disintegration rate of the tablet after oral intake. In another embodiment, a volatizable component is used to form porous tablets. Some embodiments disintegrate in the mouth in less than 10 seconds.

U.S. Pat. No. 5,382,437 to Ecanow discloses a porous carrier material having sufficient rigidity for carrying and administering an active agent which is capable of rapid dissolution by saliva. The porous carrier material of Ecanow is formed by freezing a liquified ammonia solution comprising liquid ammonia, liquid ammonia soluble gel or foam material, and a rigidifying agent for the gel or foam material selected from the group consisting of a monosaccharide, a polysaccharide and combinations thereof, and deammoniating the frozen material thus formed, by causing material transfer of ammonia from the frozen state to the gas state thereby leaving spaces in the carrier material in place of the frozen ammonia.

U.S. Pat. No. 5,631,023 discloses a method for manufacturing a therapeutic tablet that dissolves nearly instantaneously upon contact with water. This method incorporates a particular agent into an aqueous gelatin containing suspension in order to keep a granular therapeutic agent uniformly dispersed. The method includes the preparation of a liquid admixture comprising a solvent, gelatin, a granular therapeutic agent having a particle size ranging from about 1 to about 400 microns and from 0.01 to 0.05 weight percent xanthan gum sufficient to act predominantly as a gelatin flocculating agent; filing said liquid admixture into one or more shaped depressions in a tray; freezing said liquid admixture in said trays so as to form solid shaped admixtures of solvent, carrier and granular therapeutic agent; and removal of said solvent so as to form a solid shaped tablet of carrier matrix and granular therapeutic agent. Resulting solid, shaped dosage forms are said to disintegrates in less than 10 seconds upon contact with an aqueous media.

U.S. Pat. Publication No. 20040156894 discloses a multi-phasic, lyophilized, fast-dissolving dosage form (FDDF) for the delivery of a pharmaceutically active ingredient is prepared by sequential dosing of a formulation containing a non-gelling matrix forming agent and a formulation containing a gelling gelatin.

U.S. Pat. Application No. 20110229573 discloses a FDDF process of manufacturing includes the sequential steps of: (a) dosing a formulation comprising a non-gelling matrix forming agent into a preformed mold; (b) dosing a formulation comprising a gelling matrix forming agent into the preformed mold; and (c) freeze drying the formulations dosed in steps (a) and (b) to form the multi-phasic, fast-dissolving dosage form.

U.S. Pat. No. 4,642,903 discloses a method of preparing a freeze-dried foam including an active ingredient, such as a pharmaceutical, nutrient, diagnostic, insecticide or fertilizer. The method of preparing includes forming a dispersion of a gas and a solution or suspension, said solution or suspension containing the active ingredient dissolved or suspended therein; maintaining the gas in a dispersed state within the dispersion; and freeze-drying a unit volume to form a freeze-dried foam containing the active ingredient dispersed therethrough.

U.S. Pat. No. 5,188,825 discloses a method of preparing a freeze-dried dosage form including a water soluble active agent. The water soluble active agent is bonded to an ion exchange resin to form a substantially water insoluble complex. This complex is then mixed with a compatible carrier and freeze-dried. The method involves freeze-drying an aqueous suspension consisting essentially of a) a substantially water insoluble bound bioactive agent complex consisting essentially of the bioactive agent bound to an ion exchange resin and b) an aqueous carrier compatible with the bioactive agent consisting essentially of water and a bulk-forming agent selected from the group consisting of gelatin, polyvinylpyrrolidone, polyethylene glycol, polysaccharides, and combinations thereof.

U.S. Pat. No. 5,976,577 discloses a process for preparing an oral solid, rapidly disintegrating freeze-dried dosage form of a pharmaceutically active substance having an unacceptable taste, wherein prior to freeze drying, a suspension of uncoated or coated coarse particles of a pharmaceutically active substance in a carrier material is cooled to reduce the viscosity and minimize release of the active substance during processing, and to minimize bad taste from the drug when administered.

U.S. Pat. No. 9,775,819 discloses an oral solid dosage form containing nanoparticles that is made by (a) reducing the particle size of at least one pharmaceutically active ingredient dispersed in a solution containing fish gelatin to form a nanosuspension and (b) freeze-drying the nanosuspension of step (a) to form the oral solid dosage form.

U.S. Pat. Application Publication No. 20040156894 discloses using edible acid, such as citric acid, in a formulation to reduce the disintegration time of solid, oral, fast-dispersing, lyophilized, pharmaceutical dosage forms having a pharmaceutically active ingredient with low water solubility.

U.S. Pat. Application Publication No. 20060233873 discloses a dispersion of coated crystals or granules of active substance in a lipophilic vehicle for taste masking in chewable or fast dissolving soft gelatin capsules.

U.S. Pat. Application Publication No. 20040156894 discloses a process for the preparation of a rapidly disintegrating dosage form a pharmaceutically active substance which has an unacceptable taste wherein there is formed a solution or a suspension in a solvent of a form of the pharmaceutically active substance which is less soluble in water and more palatable than the form with the unacceptable taste together with a water-soluble or water-dispersible carrier material. Discrete units of the suspension or solution are formed and the solvent is removed from the discrete units under conditions whereby a network of the carrier material carrying a dosage for the less soluble and more palatable form of the pharmaceutically active substance is formed.

Different packaging components of lyophilized products have been disclosed. For instance, U.S. Pat. No. 6,890,472 relates to a method and apparatus for forming (or cold-forming) an embossed blister from a laminated film wherein an indicia is formed on the base of the blister. In particular, a single pass process of combining the formation of a blister and indicia (embossing) on the blister is taught where a blister-forming pin contains a face with an indicia and is adapted to controllably stretch the laminated film during blister formation to minimize stretching of the film at the base of the blister.

U.S. Pat. No. 5,729,958 discloses an improved method for manufacturing freeze dried pharmaceutical tablets in blister packs. Liquid dosages are introduced into a multilayer laminated blister sheet having an impermeable intermediate layer that is positioned between first and second outer layer, each of which has substantially the same coefficient of thermal expansion. Following the introduction of the dosages into the depressions of the blister sheet, the dosages are frozen and freeze dried. A lidding sheet is then attached to the blister sheet to seal the solid dosages into the blister pack.

U.S. Pat. No. 6,391,237 discloses a method of forming a laminated film comprising a metal foil and a polymeric layer on either side of the foil with at least one blister the base of which bears projecting indicia for molding into a body cast therein. The method comprises cold-forming the blister by advancing a pin in a direction transversely relative to the plane of the film; and stamping the indicia into the base of the blister so formed by advancing a die in the opposite direction against a mold held against the inner face of the blister base.

U.S. Pat. No. 6,588,180 discloses a blister pack wherein the blister includes a protruding region between the opening and the base, producing a constricted area or “neck” in the blister. The protruding region comprises an inwardly directed annulus formed in the blister wall to confine the dosage form.

Zydis® is a commercially available fast dissolving technology platform that claims dissolution of tablets in three seconds and up to ten times faster than other ODT products. Zydis® Ultra is an ODT formulation said to have increased drug loading and greater taste masking capabilities compared to conventional Zydis. Although the Zydis Ultra platform technology claims it is for higher doses>400 mg, no other information is disclosed about how it can be made and what are the properties of this dosage form.

Known issues with larger weight dosage forms (about 200 mg or greater than 200 mg) undergoing lyophilization are deformation of the lyophilized dosage form. In particular, the center core cavitates and becomes denser and thinner compared to the outer perimeter of the tablet. Additionally, the core can have uneven density in the center, which causes the tablet to take a longer time for dispersion and dissolution.

There is a desire for fast dissolving tablets that contain greater than 200 mg of active ingredient and having a center core, which do not exhibit deformation of the center core.

There is a desire for fast dispersing oral and vaginal pharmaceutical dosage forms that have an even density in their center core.

In is an object of the invention to provide improved fast dissolving pharmaceutically acceptable dosage forms containing active pharmaceutical ingredients that can be administered vaginally or orally.

There is a desire for fast dissolving pharmaceutical tablets containing greater than 200 mg of active ingredient that disintegrate in and/or have a dissolution rate of about 30 seconds or less.

It is an object of the invention to provide an improved lyophilization process for preparing fast dissolving pharmaceutically acceptable tablets containing active pharmaceutical ingredients that can be administered vaginally or orally.

### SUMMARY OF THE INVENTION

In one aspect, the invention provides a lyophilized pharmaceutical dosage form comprising a top surface, a bottom surface, a depth spanning from the top surface to the bottom surface, and a peripheral diameter, wherein the bottom surface of the tablet contains a void that is about 60% to 100% of the depth of the tablet. The dosage form is preferably a tablet for oral or vaginal administration that is rapidly dispersible, rapidly disintegrates, or rapidly dissolves when exposed to an aqueous environment.

In another aspect, the invention provides a cavity for preparing a lyophilized pharmaceutical dosage form comprising a top surface, a bottom surface, a depth spanning from the top surface to the bottom surface, and a peripheral diameter, wherein the bottom surface contains a protrusion having a height that is about 60% to 100% of the depth of the cavity.

In yet another aspect, the invention provides a base element for a lyophilization process comprising a top surface having plurality of cavities, each one of said cavities comprising a top surface, a bottom surface, a depth spanning from the top surface to the bottom surface, and a peripheral diameter, wherein the bottom surface contains a protrusion that is about 60% to 100% of the depth of the cavity.

The invention further provides a method of a manufacturing a lyophilized pharmaceutical dosage form, such as a tablet, comprising a top surface, a bottom surface, a depth spanning from the top surface to the bottom surface, and a peripheral diameter, wherein the bottom surface of the tablet contains a void that is about 60% to 100% of the depth of the tablet. The method comprises using a plurality of the aforementioned cavities and, optionally, the aforementioned base element. A blister material, such as an aluminum based material, may be placed on the element and in the cavities. A pre-lyophilization composition is filled into the cavities and the compositions are placed into a lyophilizer chamber and lyophilized using standard settings, which includes freezing, primary drying and secondary drying. A backing/overwrap material can be placed on the blister material, such that the tablets are sealed into their primary packaging during the process.

### BRIEF DESCRIPTION OF THE DRAWINGS

FIGS. 1A, 1B, and 1C show a top view, a perspective view, and a side view, respectively, of a lyophilized fast dispersing tablet of the present invention.

FIGS. 2A-2F show process steps for manufacturing a tablet of FIG. 1.

FIG. 3A shows a side view of a tablet in a blister and FIG. 3B shows a side view of a tablet without a blister.

FIG. 4 shows formation of a blister with cavities used to prepare a fast dispersible tablet by a lyophilization process.

FIG. 5 shows a perspective view of a blister with a plurality of cavities that can be used to make the tablet of FIG. 1 by a lyophilization process.

FIGS. 6A and 6B show a side view and a top view respectively of a lyophilization promoting base element that can be used to prepare fast dissolving tablets by a lyophilization process.

FIGS. 7A and 7B show a side view and a top view, respectively, of a lyophilization promoting base element that can be used in a lyophilization process to prepare the tablets of FIG. 1.

FIG. 8 is a top and side perspective view, with additional projections of a bottom view and a side view of an exemplary blister cavity used for preparing a dispersible tablet by lyophilization.

FIG. 9 is a table illustrating examples of volume obtained and contact surface area with cavity of a blister for various sized tablets of the invention, including density and respective weight.

### DETAILED DESCRIPTION OF THE INVENTION

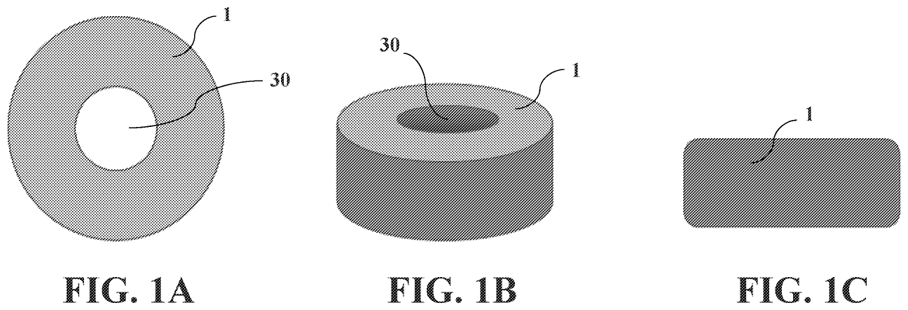

Improved fast dissolving tablets as shown in FIGS. 1A-1C comprise an active ingredient and excipients. The tablets contain one or more active ingredients combined with excipients in a pre-lyophilization composition, preferably a solution or suspension. Typically, water will be used as a solvent/vehicle for the pre-lyophilization composition. However, other solvents and co-solvent systems may be used as a vehicle and will generally be known to those of skill in the art with lyophilization processes. The tablets are prepared in cavities/blisters by a lyophilization process using a lyophilization promoting element. The cavity/blisters serve as a primary packaging for storage of the tablets.

The tablets each contain about 50-2,000 mg of solid, lyophilized material, typically 75-1,000 mg solid, lyophilized material. In certain preferred embodiments, a tablet contains greater than or equal to 200 mg solid, lyophilized material. In other preferred embodiments, a tablet contains greater than or equal to 400 mg solid, lyophilized material. In certain embodiments, a tablet contains greater than 200 mg, preferably greater than 400 mg solid, lyophilized material.

The active ingredient may be selected from any pharmaceutically acceptable agent that is suitable for manufacture by a lyophilization process.

The amount of active ingredient will depend on the active ingredient and therapeutic effect desired. The amount of active ingredient can vary from greater than zero to about 1,000 mg.

In certain embodiments, about 1 mg to about 10 mg of active ingredient is contained in a tablet.

The active ingredient is combined with non-active excipients. The excipients can include crystallization prohibitor, bulking agent, sweetener, flavoring agent, pH regulating agent, anti-oxidant, chelating agent, taste modifier, preservative or any combination thereof.

Taste modifiers used in the present invention increase patient acceptability and are selected from one or more of sweetening agents, such as monosaccharides, disaccharides, sugar alcohols, and polysaccharides, e.g., glucose, fructose, invert sugar, sorbitol, sucrose, maltose, xylose, ribose, mannose, corn syrup solids, xylitol, mannitol, maltodextrins, and mixtures thereof, artificial sweeteners and dipeptide-based sweeteners, such as saccharin salts, acesulfame K, sucralose, aspartame, and mixtures thereof.

Preservatives used in the present compositions may be selected from one or more of benzalkonium chloride, benzyl alcohol, chlorobutanol, cresol, ethyl alcohol, thiomersal, parabens, benzoic acid, EDTA, sodium benzoate and the like.

Antioxidants used in the present compositions may be selected from one or more of, e.g., sulfites, amino acids, such as L-methionine, ascorbic acid and a-tocopherol. Preferably, the antioxidant is L-methionine

Flavors, which may optionally be used in the present invention, can be selected from one or more naturally derived oils from plants, flowers, leaves, and artificial flavoring compounds, such as synthetic flavor oils.

Buffers used in the present invention can include an acid or a base and its conjugate base or acid, respectively. Suitable buffers include mixtures of weak acids and alkali metal salts (e.g., sodium, potassium) of the weak acids, such as acetate, citrate, tartrate, phosphate, benzoate and bicarbonate buffers and combinations thereof.

pH regulating agents can include buffers, such as acetate, citrate, phosphate, borate, carbonate etc., sodium hydroxide, hydrochloric acid etc.

A crystallization prohibitor can include polymers selected from the group of hydroxypropyl cellulose, hypromellose, polyvinyl pyrrolidone, carboxymethyl cellulose sodium, Carbopol, alginic acid or its sodium salt, cellulose, cellulose acetate, polyethylene glycol, crospovidone, copovidone and combinations thereof.

An exemplary pre-lyophilization composition for preparing a lyophilized orally dispersing tablet comprises an active pharmaceutical agent; a polymer selected from the group consisting of gelatin, hydroxypropyl cellulose, polyvinyl pyrrolidone, carboxymethyl cellulose or its salts, croscarmellose sodium, cellulose derivative polymer and sugar polymers, and solvent. The pre-lyophilization composition optionally contains one or more of Bulking agent, Sweetener, Flavoring agent, pH adjustant and/or buffering agent.

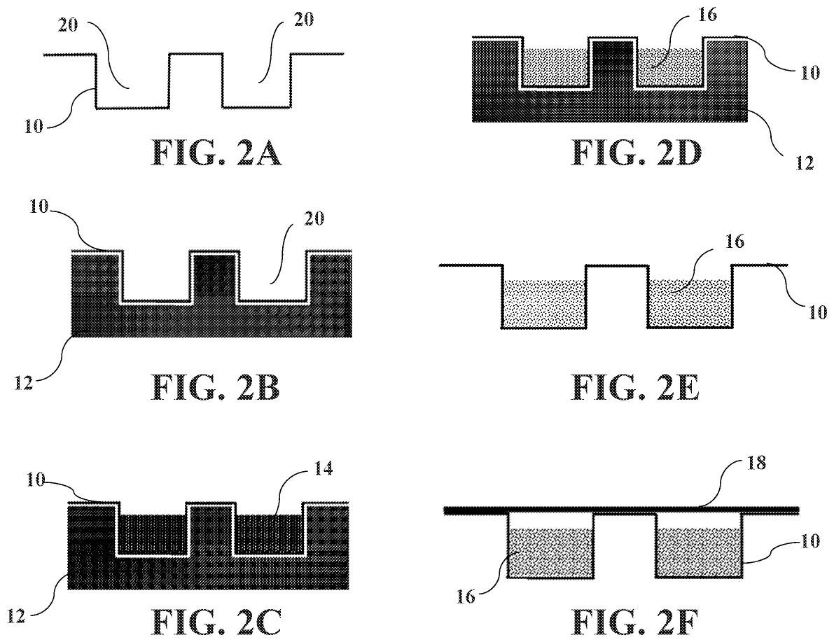

Tablet 1 shown in FIGS. 1A-1C is prepared by a lyophilization method as depicted in FIGS. 2A-2F. Lyophilization essentially consists of three steps, i.e. (i) freezing; (ii) primary drying; and (iii) secondary drying. The pre-lyophilization composition 14 is placed into a cavity 20 in blister 10, which is preferably located in a base element 12. In the freezing step, a pre-lyophilization composition 14 is cooled below 0° C., preferably below −20° C., more preferably below −25° C. In primary drying, the composition 14 is exposed to reduced pressure about less than 800 mTorr, preferably less than 500 mTorr, more preferably about 200 mTorr and temperature is slowly increased from the previously set temperature in the freezing step. The increase in temperature is increased by about 2° C. from the temperature set in the freezing step, preferably about 5° C. In secondary drying, temperature is increased further from the previously set temperature in the primary drying step. The increase in temperature is about 5° C. from the temperature set in the primary drying step, preferably about 10° C., more preferably about 20° C. Pressure can be optionally increased or decreased during secondary drying.

FIG. 2A shows an aluminum “blister” 10 (often called a “blister pack”) that forms the cavities 20, which may also serve as the primary packaging for the tablets 1. FIG. 2B shows the blister 10 placed on a lyophilization promoting base element 12 for improved lyophilization. Pre-lyophilization solution 14 is filled in the cavities 20 formed in the blister 10, as shown in FIG. 2C. Next, the lyophilization process is performed and solvent is removed, leaving a uniform “cake” 16 which comprises the tablet 1, as illustrated in FIG. 2D. Then, the blister 10 is removed from the base element 12, as shown in FIG. 2E. Finally, the blister 10 is closed with over wrap film 18, e.g. aluminum or polymer or combined laminated material, as shown in FIG. 2F.

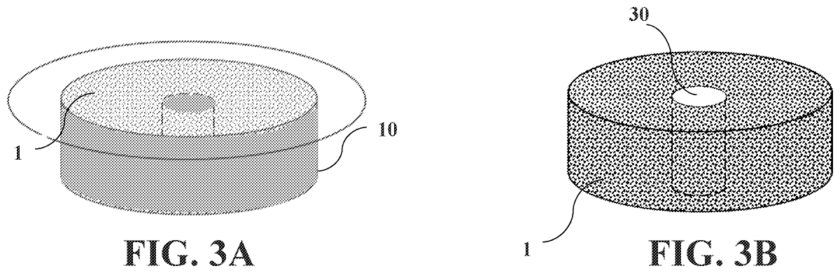

FIG. 3A shows a perspective side view of a manufactured tablet 1 wrapped in aluminum blister 10 and FIG. 3B shows a perspective side view of the tablet 1 without the blister. FIG. 3B shows tablet 1 with a void 30 in the center of the tablet 1.

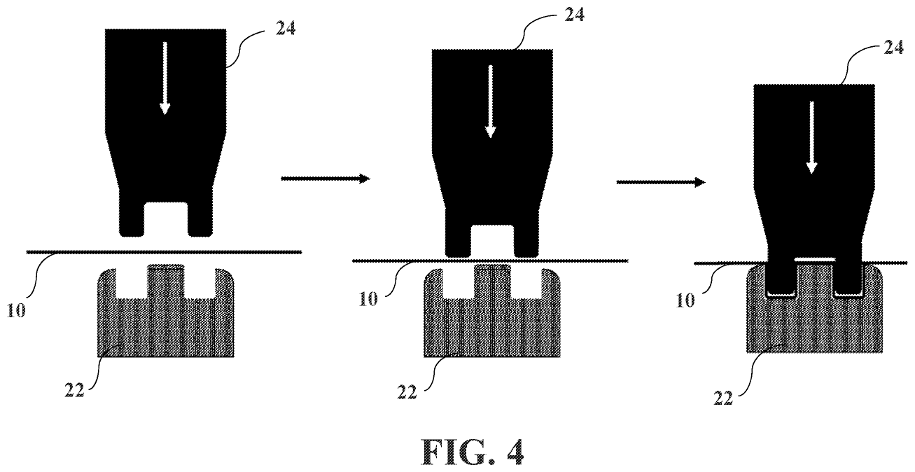

FIG. 4 shows a process for preparing a cavity 20 that can be used in the process of FIGS. 2A-2F. In this embodiment, the cavity 20 is formed from an aluminum blister sheet. A blister sheet is placed between two parts of a stamping tool. Other deformable materials for forming the cavity 20 are contemplated and within the scope of the invention. It is preferred that the cavity 20 is formed from a material that can protect the lyophilized tablet from moisture while being stored. A bottom part 22 of the stamping tool has a shape that corresponds to the desired shape of the cavity. A top part 24 of the stamping tool corresponds to the shape of the bottom part 22. Once the blister sheet 10 is placed between the top and bottom parts 24, 22, the top part 24 is moved downward and into the mating cavity of the bottom part 22 and the blister sheet is compressed between the two parts thereby forming a blister 10 with one or more cavities 20 in the blister 10. The person skilled in the art of pharmaceutical packaging will be aware of the many other processes and materials that are suitable for forming the cavities.

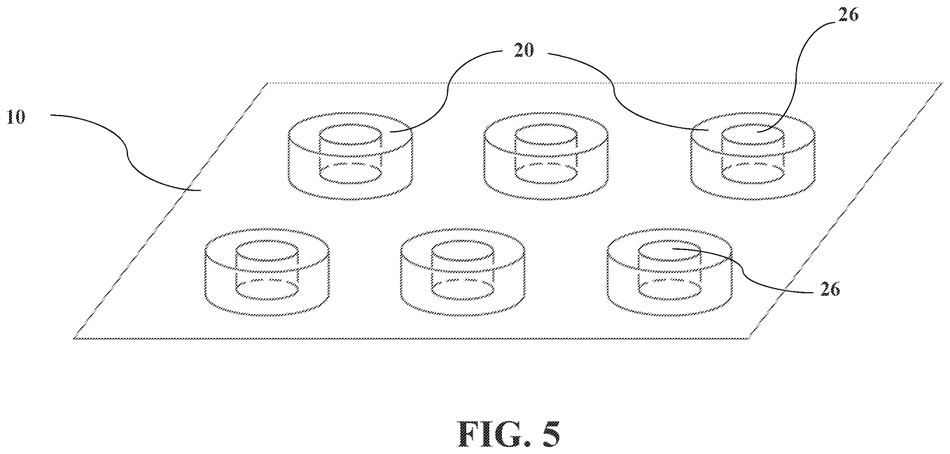

FIG. 5 depicts a blister 10 with a plurality of cavities 20 that can be used in the process of FIGS. 2A-2F. In this embodiment, the cavities 20 are round with a protrusion 26 in the center, such that a tablet will be produced having an annular, donut like shape. In some embodiments, the protrusion 26 is cylindrical, or it may be a tapered cylinder, having a larger diameter at its lower end and a smaller diameter at its upper end, and the void 30 in tablet 1 will have a corresponding tapered volume, to facilitate release of the tablet 1 from its cavity 20. In some embodiments, a very highly tapered cylindrical protrusion 26 will have a truncated conical shape, and the void 30 in tablet 1 will have a corresponding truncated conical shape.

Other blister cavity protrusion 26 configurations are within the scope of the invention, for example polygonal cross-section protrusions 26 that are three, four, five, six, seven or eight sided, and which may or may not be tapered (for example, pyramidal-shaped protrusions).

The protrusion 26 in the cavity creates a male molding component around which the tablet 1 forms during lyophilization. The final dosage will have a void that corresponds in shape to the protrusion 26.

In some embodiments, the cavity may contain multiple protrusions 26 of the same or varying shapes.

The described shape of cavity 20 with protrusion 26 is advantageous in that it allows close proximity of cooling or heating elements to the pre-lyophilization composition 14 to provide efficient freeze drying of the composition. Furthermore, trademarks or patterns may be embossed on the protrusion 26 which then will appear on the finished tablet 1, to provide visual indications in the finished tablet 1 that the product is genuine and not counterfeit. Preferably, the inclusion of a protrusion to modify the cavity shape will increase the contact surface are during lyophilization by about 5% to about 30%, preferably about 10% to about 25%, most preferably about 15% to about 20%.

In preferred embodiments, the blister 10 with cavities 20 will be placed on a lyophilization promoting base element 12 prior to filling the pre-lyophilization composition. As shown in FIG. 2B, an aluminum blister 10 is seated in a base element 12 having indentations with shapes which align with and receive the cavities 20 and protrusions 26 of blister 10.

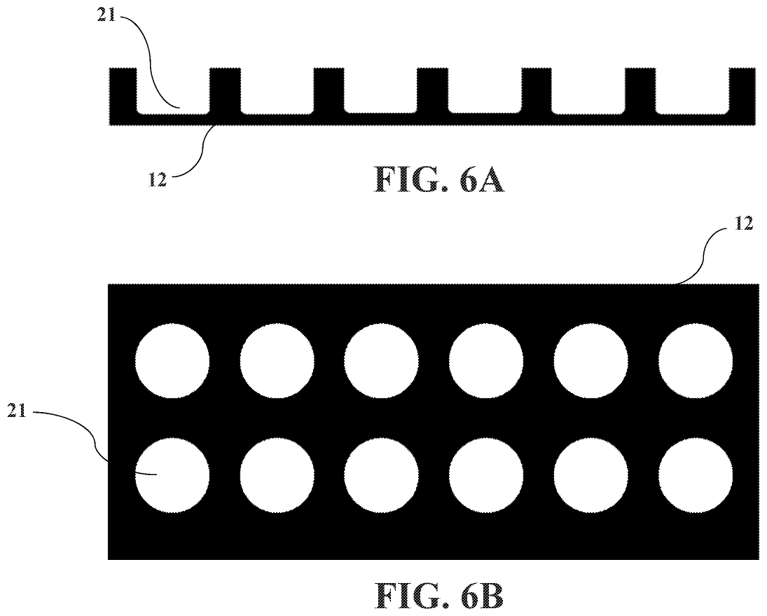

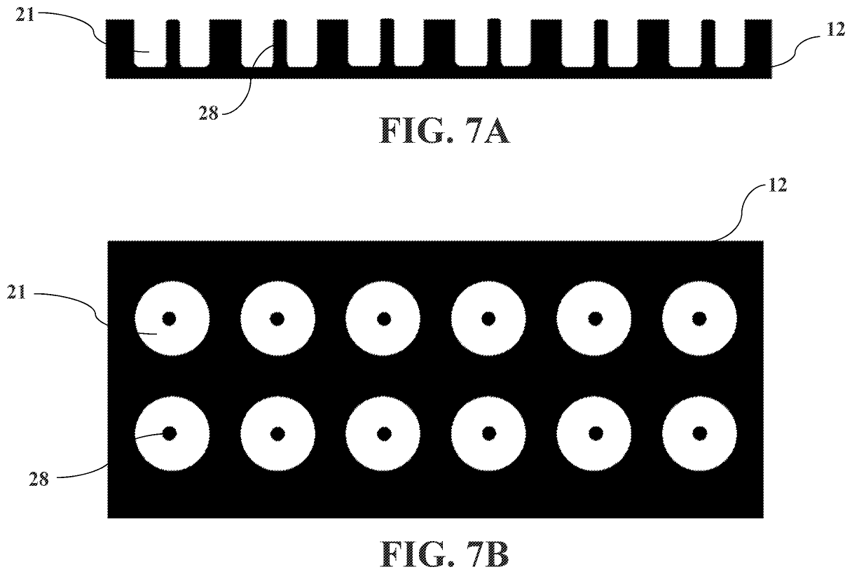

Exemplary lyophilization promoting base elements 12 that can be used in the lyophilization process are shown in FIGS. 6A-6B and 7A-7B. The lyophilization promoting base elements 12 are an efficient solution for an overall improved lyophilization process with efficient heat transfer and less tablet to tablet variation in the drying process.

The base elements 12 contain a plurality of base cavities 21, which are preferably round shaped, having a top surface, bottom surface, a depth spanning from the top surface to the bottom surface and a peripheral diameter that are used to receive a blister 10 and cavity 20 to prepare a fast dissolving dosage form having a corresponding shape.

In some embodiments, the base element 12 comprises a thermally conductive material with a thermal conductivity coefficient λ of about 0.1 to about 400.0 [W/mK] at 20° C. at 1 bar and a co-efficient of linear thermal expansion a of about 1 to about 25 [10⁻⁶° C.⁻¹] at normal temperature.

In certain embodiments, the lyophilization promoting base element 12 is comprised of material selected from aluminum, copper, iron, bronze, silicon, germanium, antimony, cadmium, cesium, chromium, cobalt, silver, gold, titanium, platinum, carbon and combinations thereof, oxides thereof, or alloys thereof.

In some preferred embodiments, the base element 12 is comprised of aluminum or oxides of aluminum having high heat conductivity.

In certain embodiments, the base element 12 is comprised of a hollow polymeric material and a fluid with a negative thermal expansion property.

In an embodiment shown in FIGS. 7A-7B, the base element 12 contains a plurality of round shaped base cavities 21 having a top surface, bottom surface, a depth spanning from the top surface to the bottom surface and a peripheral diameter that are used to prepare a fast dissolving dosage form. The peripheral diameter of the base cavity 21 is about 0.5 cm to about 4 cm and depth of about 0.5 cm to 4 cm. The base cavity 21 has a base protrusion 28 on its bottom surface having a height that is about 60% to 100% of the depth of the base cavity 21. The diameter of the base protrusion 28 is about 10% to 60% of the peripheral diameter of the base cavity 21. Preferably, the base protrusion 28 is located in the center of the bottom surface of each base cavity 21.

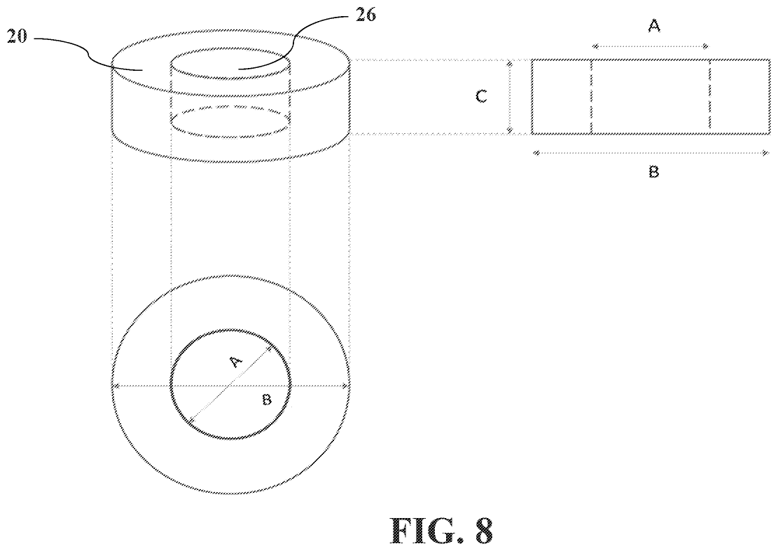

FIG. 8 shows an exemplary cavity 20 having a protrusion 26 extending upwardly from a floor of the cavity 20 that can be placed over base protrusion 28 of the base element 12 of FIG. 7B. The protrusion 26 has a diameter A or other maximum dimension that is about 10% to 60% of the diameter B of the cavity 20. The depth of the protrusion 26 is substantially the same as the cavity 20 depth C. However, other protrusion 26 depths are within the scope of the invention. Typically, the protrusion 26 depth is about 60% to 100% of the depth C of the cavity 20. Preferably, blister cavity 20 is about 0.5 cm to about 4 cm in diameter and has a depth of about 0.5 cm to 4 cm. Void 30 in tablet 1 has a shape which conforms to the protrusion 26.

Preferably, the peripheral walls of each base cavity 21 in the base element 12 and the shape of the blister cavity 20 are sized and shaped so that there is a space of no more than 0.5 millimeters between an outer wall of an inserted blister cavity 20 and the base cavity 21 peripheral walls.

In some embodiments, the base element 12 has a plurality of base cavities 21 having base protrusions 28 and the cavities 20 and protrusions 26 in blister sheet 10 are formed by placing blister sheet 10 on the base element 12 and pressing the blister sheet 10 onto base element 12 to conform the blister sheet 10 to the base element 12.

The volume of pre-lyophilization solution filled into a cavity 20 is typically about 0.25 mL to about 15 mL, more preferably about 0.50 mL to about 15 mL, most preferably about 0.75 to about 10 mL. The density of the pre-lyophilization solutions will vary depending on the active ingredient(s) but is typically from about 0.1 g/mL to about 1.0 g/mL, more preferably about 0.2 g/mL to about 0.5 g/mL.

Typically, a tablet made using the process and apparatus described herein will weigh about 0.1 grams to about 10 grams, more preferably about 0.2 grams to about 7 grams, most preferably about 0.25 grams to about 6.5 grams. The weight of the tablet within these ranges will depend on the active ingredient(s) used and the method of treatment.

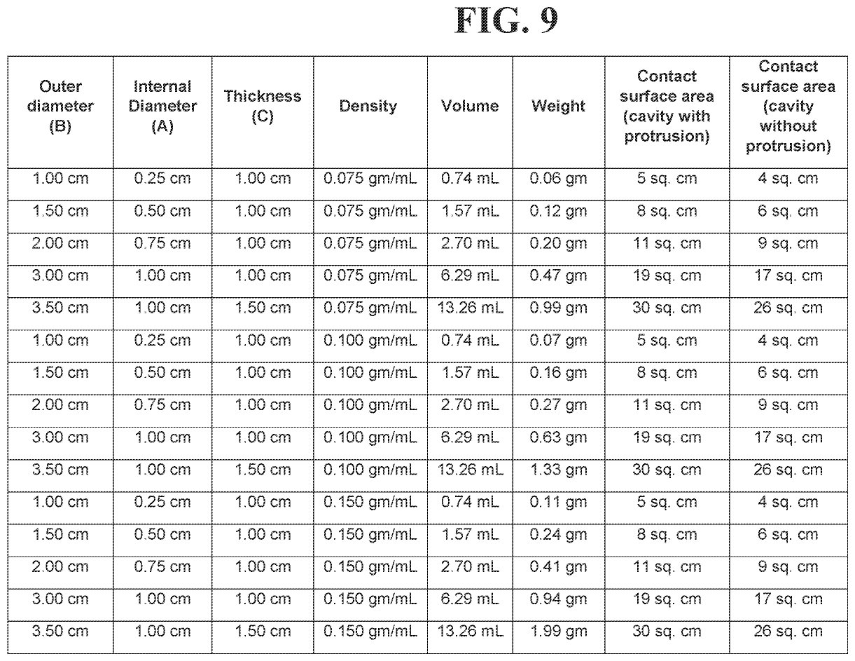

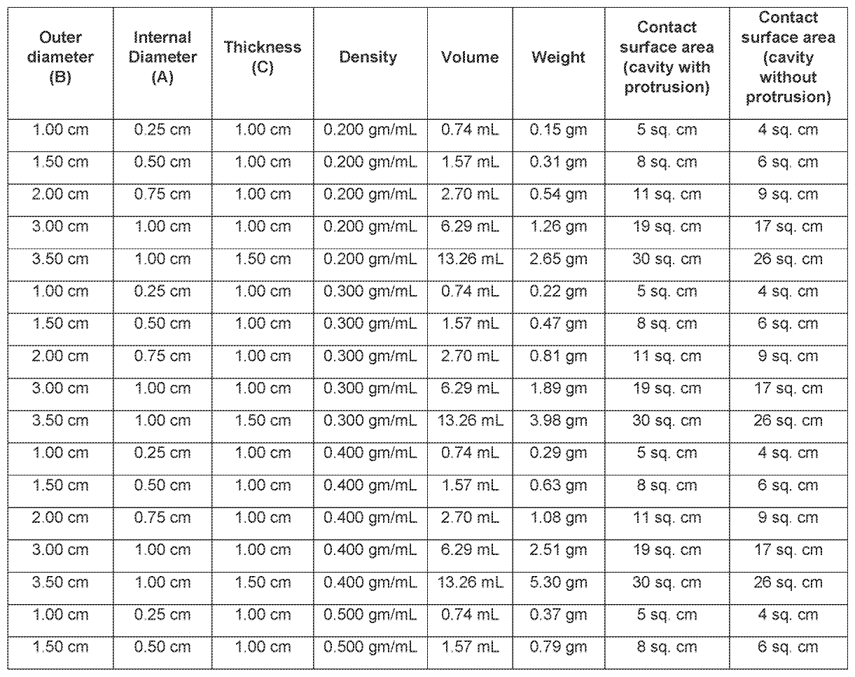

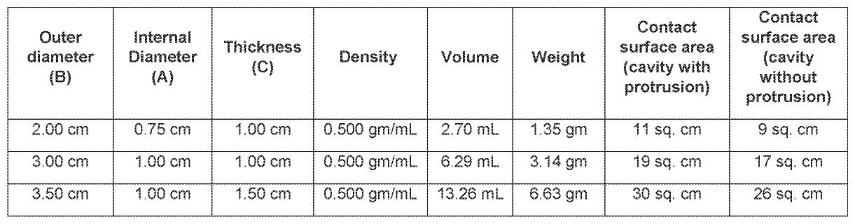

The table in FIG. 9 lists proposed tablet dimensions for tablets 1 which could be made in accordance with the invention. In the table of FIG. 9, Outer Diameter B corresponds to a tablet 1 outer diameter, Internal Diameter A corresponds to a tablet 1 void 30 diameter, and Thickness C corresponds to a tablet 1 thickness. A calculated tablet volume, and tablet weight are provided. In the table of FIG. 9, the calculated contact surface area between a prelyophilization solution 14 and a cavity 20 is calculated for a cavity 20 with a protrusion 26 and also for a cavity without a protrusion 26. In general, the contact surface area for a cavity 20 with a protrusion 26 will be 14%-20% greater than in the cavity 20 without a protrusion 26.

The invention is especially suited to provide orally disintegrating or dissolving (or both) tablets prepared by lyophilization. The following examples are proposed formulations of products particularly suitable for manufacturing using the above described processes to create a tablet form. Examples with Asenapine as the model active are provided.

### EXAMPLES

Example 1: Composition of pre-lyophilization solution for orally disintegrating tablet of Asenapine prepared by lyophilization with density of about 0.075 gm/mL.

| Sr. No. | Ingredients | Quantity/mL | | Function |
|---|---|---|---|---|
| 1 | Asenapine maleate | 5 | mg | Active |
| 2 | Hydroxypropyl cellulose | 25 | mg | Crystallization |
| | | | | prohibitor |
| 3 | Mannitol | 25 | mg | Bulking agent |
| 4 | Sucralose | 18 | mg | Sweetener |
| 5 | Citric acid | 2 | mg | Flavoring agent |
| 6 | Water (removed during | Q.s. to 1 mL | | Vehicle |
| | lyophilization process) | | | |

Example 2: Composition of pre-lyophilization solution for orally disintegrating tablet of Asenapine with density about 0.100 gm/mL

| Sr. No. | Ingredients | Quantity/mL | | Function |
|---|---|---|---|---|
| 1 | Asenapine maleate | 5 | mg | Active |
| 2 | Hydroxypropyl cellulose | 37.5 | mg | Crystallization |
| | | | | prohibitor |
| 3 | Mannitol | 37.5 | mg | Bulking agent |
| 4 | Sucralose | 18 | mg | Sweetener |
| 5 | Citric acid | 2 | mg | Flavoring agent |
| 6 | Water (removed during | Q.s. to 1 mL | | Vehicle |
| | lyophilization process) | | | |

Example 3: Composition of pre-lyophilization solution for orally disintegrating tablet of Asenapine with density about 0.150 gm/mL

| Sr. No. | Ingredients | Quantity/mL | | Function |
|---|---|---|---|---|
| 1 | Asenapine maleate | 5 | mg | Active |
| 2 | Hydroxypropyl cellulose | 62.5 | mg | Crystallization |
| | | | | prohibitor |
| 3 | Mannitol | 62.5 | mg | Bulking agent |
| 4 | Sucralose | 18 | mg | Sweetener |
| 5 | Citric acid | 2 | mg | Flavoring agent |
| 6 | Water (removed during | Q.s. to 1 mL | | Vehicle |
| | lyophilization process) | | | |

Example 4: Composition of pre-lyophilization solution for orally disintegrating tablet of Asenapine with density about 0.200 gm/mL

| Sr. No. | Ingredients | Quantity/mL | | Function |
|---|---|---|---|---|
| 1 | Asenapine maleate | 5 | mg | Active |
| 2 | Hydroxypropyl cellulose | 87.5 | mg | Crystallization |
| | | | | prohibitor |
| 3 | Mannitol | 87.5 | mg | Bulking agent |
| 4 | Sucralose | 18 | mg | Sweetener |
| 5 | Citric acid | 2 | mg | Flavoring agent |
| 6 | Water (removed during | Q.s. to 1 mL | | Vehicle |
| | lyophilization process) | | | |

Example 5: Composition of pre-lyophilization solution for orally disintegrating tablet of Asenapine with density about 0.200 gm/mL

| Sr. No. | Ingredients | Quantity/mL | | Function |
|---|---|---|---|---|
| 1 | Asenapine maleate | 5 | mg | Active |
| 2 | Hydroxypropyl cellulose | 50 | mg | Crystallization |
| | | | | prohibitor |
| 3 | Mannitol | 125 | mg | Bulking agent |
| 4 | Sucralose | 18 | mg | Sweetener |
| 5 | Citric acid | 2 | mg | Flavoring agent |
| 6 | Water (removed during | Q.s. to 1 mL | | Vehicle |
| | lyophilization process) | | | |

Example 6: Composition of pre-lyophilization solution for orally disintegrating tablet of Asenapine with density about 0.150 gm/mL

| Sr. No. | Ingredients | Quantity/mL | | Function |
|---|---|---|---|---|
| 1 | Asenapine maleate | 5 | mg | Active |
| 2 | Polyvinyl pyrrolidone | 20 | mg | Crystallization |
| | | | | prohibitor |
| 3 | Lactose | 105 | mg | Bulking agent |
| 4 | Sucralose | 18 | mg | Sweetener |
| 5 | Citric acid | 2 | mg | Flavoring agent |
| 6 | Water (removed during | Q.s. to 1 mL | | Vehicle |
| | lyophilization process) | | | |

Additional examples are given for Carglumic acid fast disintegrating or dissolving tablets as following.

Example 7: Composition of lyophilized fast dispersing and/or dissolving tablet of Carglumic acid, 1.8 gm for oral administration.

| Sr. No. | Ingredients | Quantity/mL | | Function |
|---|---|---|---|---|
| 1 | Carglumic acid | 1.8 | gm | Active |
| 2 | Hydroxypropyl cellulose | 200 | mg | Crystallization |
| | | | | prohibitor |
| 3 | Water (removed during | Q.s. to 4 mL | | Vehicle |
| | lyophilization process) | | | |

Example 8: Composition of lyophilized fast dispersing and/or dissolving tablet of Carglumic acid, 1.8 gm for oral administration.

| Sr. No. | Ingredients | Quantity/mL | | Function |
|---|---|---|---|---|
| 1 | Carglumic acid | 1.8 | gm | Active |
| 2 | Sodium lauryl sulfate | 4.5 | mg | Surfactant |
| 3 | Cross carmellose | 200 | mg | Crystallization |
| | sodium | | | prohibitor/ |
| | | | | disintegrating agent |
| 4 | Water (removed during | Q.s. to 4 mL | | Vehicle |
| | lyophilization process) | | | |

Example 9: Lyophilization cycle of pre-lyophilization solution of orally disintegrating Asenapine tablets filled in aluminum blister with 1 mL fill volume.

| | | | Ramp Time | Hold time | |
|---|---|---|---|---|---|
| Stage | Temperature | | (min) | (min) | Vacuum |
| Freezing | −45° | C. | 150 | 300 | — |
| Primary Drying | −45° | C. | — | 10 | 100 mT |
| | −20° | C. | 250 | 1000 | 100 mT |
| | −5° | C. | 250 | 120 | 100 mT |
| Secondary Drying | 25° | C. | 375 | 600 | 100 mT |

Example 10: Lyophilization cycle of pre-lyophilization solution of orally disintegrating Asenapine tablets filled in aluminum blister with 1 mL fill volume (with annealing step during freezing).

| | | | Ramp Time | Hold time | |
|---|---|---|---|---|---|
| Stage | Temperature | | (min) | (min) | Vacuum |
| Freezing | −45° | C. | 150 | 120 | — |
| | −25° | C. | 60 | 180 | — |
| | −45° | C. | 60 | 180 | — |
| Primary Drying | −45° | C. | — | 10 | 100 mT |
| | −20° | C. | 250 | 1000 | 100 mT |
| | −5° | C. | 250 | 120 | 100 mT |
| Secondary Drying | 25° | C. | 375 | 600 | 100 mT |

Example 11: Lyophilization cycle of pre-lyophilization solution of fast disintegrating Carglumic acid tablets filled in aluminum blister with 4 mL fill volume (with annealing step during freezing).

| | | | Ramp Time | Hold time | |
|---|---|---|---|---|---|
| Stage | Temperature | | (min) | (min) | Vacuum |
| Freezing | −45° | C. | 150 | 120 | — |
| | −25° | C. | 60 | 180 | — |
| | −45° | C. | 60 | 240 | — |
| Primary Drying | −45° | C. | — | 10 | 100 mT |
| | −20° | C. | 250 | 1500 | 100 mT |
| | −5° | C. | 250 | 120 | 100 mT |
| Secondary Drying | 25° | C. | 375 | 600 | 100 mT |

While the present teachings have been described above in terms of specific embodiments and examples, it is to be understood that they are not limited to those disclosed embodiments and examples. Many modifications to the embodiments and examples will come to mind to those skilled in the art to which this pertains, and which are intended to be and are covered by both this disclosure and the appended claims. It is intended that the scope of the present teachings should be determined by proper interpretation and construction of the appended claims and their legal equivalents, as understood by those of skill in the art relying upon the disclosure in this specification and the data tables.

What is claimed is:

1. A pharmaceutical dosage form comprising:
a tablet formed from a pre-lyophilization solution, said tablet having a top surface, a bottom surface, and a void extending from the bottom surface of the tablet toward the top surface of the tablet, said void having a height that is about 60% to 100% of a depth of the tablet.

2. The pharmaceutical dosage form of claim 1, wherein the void is round.

3. The pharmaceutical dosage form of claim 1, wherein the dosage form is contained in a cavity in a blister sheet.

4. The pharmaceutical dosage form of claim 3, further comprising an over wrap film sealing the dosage form in the cavity in the blister sheet.

5. The pharmaceutical dosage form of claim 4, wherein a plurality of the dosage forms is sealed in a plurality of cavities in the blister sheet.

6. The pharmaceutical dosage form of claim 1, wherein the void is a cylinder or tapered cylinder.

7. A pharmaceutical dosage form, comprising:
a blister sheet having one or more cavities having a diameter and a depth, at least one of said one or more cavities having a protrusion extending upwardly from a floor of said cavity, said protrusion having a maximum dimension which is about 10% to 60% of the diameter of the cavity, and having a depth which is about 60% to 100% of the depth of the cavity;
a tablet formed in the cavity having a protrusion by lyophilization of a pre-lyophilization solution, said tablet having a top surface, a bottom surface, and a void extending from the bottom surface of the tablet toward the top surface of the tablet, said void having a shape conforming to the protrusion.

8. The pharmaceutical dosage form of claim 7, wherein the protrusion is a cylinder or tapered cylinder.

9. The pharmaceutical dosage form of claim 7, further comprising an over wrap film sealing the tablet in the blister sheet cavity.

10. The pharmaceutical dosage form of claim 7, wherein the blister sheet has more than one said cavity, and each said cavity contains a tablet formed in the cavity by lyophilization of a pre-lyophilization solution.

11. A method of manufacturing a solid pharmaceutical dosage form, comprising:
forming a blister sheet having at least one cavity having a diameter and a depth, said at least one cavity having a protrusion extending upwardly from a floor of said cavity, said protrusion having a maximum dimension which is about 10% to 60% of the diameter of the cavity, and having a depth which is about 60% to 100% of the depth of the cavity;
filling a pre-lyophilization composition into the at least one cavity;
lyophilizing the pre-lyophilization solution to form a tablet in the at least one cavity, the tablet having a top surface, a bottom surface, and a void extending from the bottom surface of the tablet towards the top surface of the tablet, said void having a shape conforming to the protrusion.

12. The method of claim 11, wherein the blister sheet is seated on a thermally conductive base element having one or more base cavities which conform to the at least one cavity in the blister sheet prior to lyophilizing the pre-lyophilization solution.

13. The method of claim 12, wherein the blister sheet cavity conforms to the one or more base cavities of the base element such that there is a space of no more than 0.5 millimeters between an outer wall of an inserted blister cavity and a peripheral wall of a base cavity containing the inserted blister cavity.

14. The method of claim 12, wherein the base element has one or more base cavities having base protrusions therein and the blister sheet is pressed onto the base element to form the at least one cavity and upwardly extending protrusion in the blister sheet.

15. The method of claim 12, further comprising sealing the tablet in the blister sheet cavity with an over wrap film.

16. The method of claim 12, wherein the blister sheet has more than one cavity and the base element has more than one base cavity.

17. A pharmaceutical packaging, comprising:
a blister sheet having at least one cavity having a diameter and a depth, said at least one cavity having a protrusion extending upwardly from a floor of said cavity, said protrusion having a maximum dimension which is about 10% to 60% of the diameter of the cavity, and having a depth which is about 60% to 100% of the depth of the cavity; and
an over wrap film sealed to the blister sheet and covering the at least one cavity.
